(19) United States
(12) Patent Application Publication
(10) Pub. No.: US 2009/0252791 A1
(43) Pub. Date: Oct. 8, 2009

(54) PHARMACEUTICAL COMPOSITIONS COMPRISING A TRIPTAN AND A NONSTEROIDAL ANTI-INFLAMMATORY DRUG

(76) Inventors: Venkata Nookaraju Sreedharala, Hyderabad (IN); Atul Vishvanath Patil, Belgaum (IN); Pramod Kharwade, Chhindwara (IN); Narayanan Badri Vishwanathan, Chennai (IN); Harshal Prabhakar Bhagwatwar, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/417,005

(22) Filed: Apr. 2, 2009

Related U.S. Application Data
(60) Provisional application No. 61/075,421, filed on Jun. 25, 2008.

(30) Foreign Application Priority Data
Apr. 2, 2008 (IN) 833/CHE/2008

(51) Int. Cl. A61K 9/20; A61K 31/404

(52) U.S. Cl. 424/464; 514/415

(57) ABSTRACT

Dosage forms for administering a triptan and a nonsteroidal anti-inflammatory drug are disclosed, including multilayered tablets and mini-tablets. These multilayered tablets can be in a side-by-side arrangement, can be inlayed, or one layer can completely surround another. These can be administered in a capsule or directly. Other formulations are also disclosed.

The present invention relates to pharmaceutical compositions comprising a triptan and a nonsteroidal anti-inflammatory drug. In certain embodiments, the present invention relates to fixed dose pharmaceutical compositions comprising sumatriptan, or any pharmaceutically acceptable salts thereof, and naproxen, or any pharmaceutically acceptable salts thereof.

Sumatriptan is a selective 5-hydroxytryptamine₁ receptor subtype agonist. Sumatriptan has a chemical name 3-[2-(dimethylamino)ethyl]-N-methyl-1H-indole-5-methanesulfonamide, and structure (1).

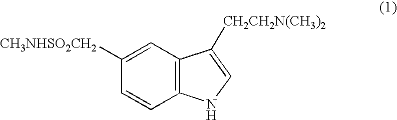

The empirical formula is C₁₄H₂₁N₃O₂S, representing a molecular weight of 295.4. Sumatriptan is a white to off-white powder that is readily soluble in water and in saline. Sumatriptan is commercially available as IMITREX™ by GlaxoSmithKline, in the form of a nasal spray delivering 5 or 20 mg of sumitriptan/100 μL unit dose.

Sumatriptan succinate has a chemical name 3-[2-(dimethylamino)ethyl]-N-methyl-indole-5-methanesulfonamide succinate (1:1), and structure (2).

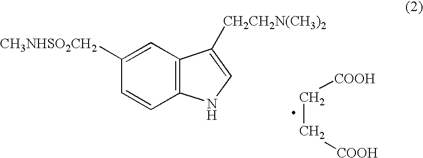

The empirical formula for sumatriptan succinate is C₁₄H₂₁N₃O₂S.C₄H₆O₄, representing a molecular weight of 413.5. Sumatriptan succinate is a white to off-white powder that is readily soluble in water and in saline. It is commercially available in products sold as IMITREX™ by GlaxoSmithKline as 25, 50 and 100 mg sumatriptan tablets, and 8 and 12 mg/mL sumatriptan subcutaneous injections.

Naproxen is a proprionic acid derivative related to the arylacetic acid group of certain nonsteroidal anti-inflammatory drugs.

Chemical names for naproxen and naproxen sodium, respectively, include (S)-6-methoxy-α-methyl-2-naphthaleneacetic acid, and (S)-6-methoxy-α-methyl-2-naphthaleneacetic acid, sodium salt. The compounds are represented by structure (3), where R═COOH for naproxen and R═COONa for naproxen sodium.

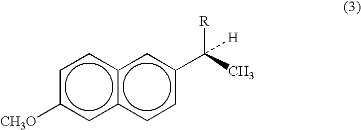

Naproxen has a molecular weight of 230.26 and a molecular formula C₁₄H₁₄O₃. Naproxen sodium has a molecular weight of 252.23 and the molecular formula C₁₄H₁₃NaO₃. Naproxen is an odorless, white to off-white crystalline substance. It is lipid-soluble, practically insoluble in water at low pH and freely soluble in water at high pH. The octanol/water partition coefficient of naproxen at pH 7.4 is 1.6 to 1.8. Naproxen sodium is a white to creamy white, crystalline solid, freely soluble in water at neutral pH. NAPROSYN™ (naproxen tablets) are available from Hoffman-La Roche as yellow tablets containing 250 mg of naproxen, pink tablets containing 375 mg of naproxen and yellow tablets containing 500 mg of naproxen, for oral administration. The inactive ingredients used in these formulations are croscarmellose sodium, iron oxides, povidone and magnesium stearate. EC-NAPROSYN™ (naproxen delayed-release tablets) is available as enteric-coated white tablets containing 375 mg and 500 mg of naproxen for oral administration. The inactive ingredients used in these formulations are croscarmellose sodium, povidone and magnesium stearate. The enteric coating contains methacrylic acid copolymer, talc, triethyl citrate, sodium hydroxide and purified water. The dissolution of this enteric-coated naproxen tablet is pH dependent with rapid dissolution above pH 6. There is no dissolution below pH 4.

ANAPROX™ (naproxen sodium tablets) is available from Hoffman-La Roche as blue tablets containing 275 mg of naproxen sodium and ANAPROX™ DS (naproxen sodium tablets) is available as dark blue tablets containing 550 mg of naproxen sodium for oral administration. The inactive ingredients are magnesium stearate, microcrystalline cellulose, povidone and talc. The coating suspension for the ANAPROX 275 mg tablet may contain hydroxypropyl methylcellulose 2910, Opaspray K-1-4210A, polyethylene glycol 8000 or Opadry YS-1-4215. The coating suspension for the ANAPROX DS 550 mg tablet may contain hydroxypropyl methylcellulose 2910, Opaspray K-1-4227, polyethylene glycol 8000 or Opadry YS-1-4216. NAPROSYN™ (naproxen suspension) is available as a light orange-colored opaque oral suspension containing 125 mg/5 mL of naproxen in a vehicle containing sucrose, magnesium aluminum silicate, sorbitol solution and sodium chloride (30 mg/5 mL, 1.5 mEq), methylparaben, fumaric acid, FD&C Yellow No. 6, imitation pineapple flavor, imitation orange flavor and purified water. The pH of the suspension ranges from 2.2 to 3.7.

Combination therapies in which triptan drugs are combined with NSAIDs for treatment of migraine patients have been reported. See for example U.S. Pat. Nos. 6,060,499; 5,872,145; 6,586,458; and 6,384,034; U.S. Published Patent Application No. 2002/0099059; EP 1051993; EP 1064967; EP 1051995; EP 10649966; and EP 1064948. In general, NSAIDs have been delivered orally, for example in the form of either single or multilayer tablets or as coated granules: U.S. Pat. Nos. 6,365,184; 5,637,320; 5,480,650; and 6,387,410. Triptans have been given orally, intranasally and by injection. See U.S. Pat. Nos. 4,816,470; 5,307,845; 5,307,953; 5,554,639; 5,705,520; and 6,368,627; and International Application Publication No. WO 00/06161. See also U.S. Patent Application Publication Nos. 2007/0184109 and 2007/0207200.

Although a work has been done in the area of dosage forms for triptans and NSAIDs, work is still needed, particularly where these agents are combined. The way in which such dosage forms are constructed may affect not only the stability of the individual therapeutic agents but also the speed at which the individual agents dissolve and become available to provide pain relief to patients.

Consider, for example, U.S. Pat. No. 7,332,183 (“'183 patent”), which is directed to a multilayer pharmaceutical tablet comprising naproxen (preferably naproxen sodium) at a therapeutic dosage of between 200 and 600 mg, and a triptan. Most preferably, the tablet has 40 mg of sumatriptan succinate, 400 mg naproxen sodium. One of the patent's central themes is that substantially all of the triptan must be found in one layer of the tablet and substantially all of the naproxen must be found in a second, separate layer. These two layers are in a side-by-side arrangement such that the dissolution of the naproxen occurs, allegedly, independently of the dissolution of triptan. This tablet arrangement allegedly has surprisingly better properties than other tablet arrangements. According to the patent, in the stomach, naproxen forms a gel-like matrix that retards the dissolution of triptans (or other drugs) unless the two agents are maintained in distinct, side by side layers.

According to the '183 patent, the term “substantially all” indicates that at least 90%, and preferably greater than 95%, of the total therapeutic agent present in the tablet is included within one distinct layer. The layers should be arranged such that the individual therapeutic agents dissolve independently of one another, i.e., dissolution should occur at approximately the same rate as would occur if the drugs were given separately. The '183 patent notes that “approximately the same rate” indicates that the time for complete dissolution of agent when drugs are given in the combination tablet should require the same amount of time ±10% as when the same amount of agent is given alone. This can allegedly be achieved by placing the individual layers in a side-by-side arrangement, as opposed to, for example, in a single layer tablet matrix containing both agents or one layer forming a core surrounded by the other layer. Again, according to the '183 patent, in a preferred embodiment, the layers are arranged so that they are juxtaposed symmetrically along a single planar surface such that essentially all of the triptan-containing layer is on one side of the plane and essentially all of the NSAID-containing layer is on the other side. The term “essentially all” in the patent is equivalent to “substantially all” and means that at least 90% and preferably greater than 95% of the therapeutic agent is present. These layers may come into direct contact with one another or, alternatively, they may be separated by one or more additional layers, e.g., a barrier layer or coating which prevents the therapeutic agents from interacting with one another.

This patent is interesting for many reasons. For one thing, it demonstrates how important the role of proper formulation can be. For another, the patent describes a bilayered tablet which seeks to segregate the naproxen and sumatriptan completely such that their rate of dissolution is independent of each other. Moreover, dissolution should continue at about the same rate for each active when compared to tablets administering the actives separately. This is a curious requirement or more correctly, the concern with the relationship of the layers, rather than the composition, is curious. Dissolution is related to a number of factors including surface area. But composition plays a big role. Moreover, the exposed surface area of each bilayer according to the patent will be significantly reduced relative to individual tablets because of the faces in intimate contact. Therefore, achieving the desired result, without a change in composition, might require an unusual tablet structure.

The patent is also interesting because it suggests that the naproxen gels and unless nearly completely segregated, the dissolution properties of the sumatriptan can be adversely affected. So complete is this teaching that the patent even suggests that nonphysical mixtures, which are nonetheless interactive, such as coating a core of one active with the other, is unacceptable. (See id. at Sections B and C, which describe comparative tablets having a naproxen core and sumatriptan film coating and a physical admixture of granulations.)

The need to so completely separate the actives adds to cost and to formulation difficulties. And, the requirement that the dissolution of the actives be virtually unchanged despite being in a multilayered tablet raises questions about the structure and architecture of the dosage forms, the types of excipients needed to accomplish this goal, and the formulation techniques necessary. Yet, if true, the '183 patent's concerns about mixtures all but preclude combination therapy using anything other than separate tablets, which again can add to cost and adds to the inconvenience of taking the medication.

A combination product of naproxen (500 mg) and sumatriptan (85 mg) is commercially available and sold as TREXIMET™ by GlaxoSmithKline. Each TREXIMET tablet for oral administration contains 119 mg of sumatriptan succinate equivalent to 85 mg of sumatriptan and 500 mg of naproxen sodium. Each tablet also contains the inactive ingredients croscarmellose sodium, dextrose monohydrate, dibasic calcium phosphate, FD&C Blue No. 2, lecithin, magnesium stearate, maltodextrin, microcrystalline cellulose, povidone, sodium bicarbonate, sodium carboxymethyl cellulose, talc, and titanium dioxide. It is believed that this formulation is intended to fall within the scope of the '183 patent.

There exists a need for an alternate design approach to compositions comprising sumatriptan and naproxen that provide desired stability and release profiles in vitro and in vivo. The present invention provides alternate design and compositions having fixed doses of sumatriptan and naproxen.

### SUMMARY

The present invention relates to pharmaceutical compositions comprising both a triptan and a nonsteroidal anti-inflammatory (NSAID). Further the present invention relates to fixed dose pharmaceutical compositions comprising a triptan, or any pharmaceutically acceptable salt thereof, and an NSAID, or any pharmaceutically acceptable salt thereof.

In an embodiment of the invention, fixed dose pharmaceutical compositions of a triptan and an NSAID are presented as layered tablets or mini-tablets. These include layers in a side-by-side arrangement. Layered tablets also include inlay tablets; wherein an “outer” tablet comprises either a triptan or NSAID and an “inner” tablet comprises a triptan when outer tablet comprises an NSAID or comprises an NSAID when the outer tablet comprises a triptan. In still another embodiment, the inner tablet is completely surrounded or coated by the outer layer or tablet. In one particular aspect of this latter embodiment, the core includes the triptan and the coating includes the NSAID.

In another embodiment of the invention, the multilayer tablet comprises a triptan or its salts and an NSAID or its salts wherein: a) between 10% or more of the triptan or its salts and up to about 90% thereof is in a first layer of the tablet together with the NSAID or its salts; and b) the remaining triptan or its salts is in a second layer of the tablet. This means that the combination layer contains an amount of triptan which is more than 10% of the total amount of triptan in the overall dosage form, but not more than about 90% thereof. The balance of the triptan is located in the second layer. Substantially all of the NSAID (except any that could migrate at the interface during tablet compression, should be disposed in the first layer. In another embodiment, the amount of triptan in the first layer is more than 10%, and in still another embodiment, 12% or more, 13% or more, 14% or more, 15% or more and 20% or more, and up to about 50%. Again, the percent reflects the percentage of, in this instance, the triptan in the first layer relative to the total amount of triptan in the dosage form. The percentages have the same overall meaning when it is the NSAID that is distributed in two layers. The layers may be in a side-by-side, inlay, or coated arrangement. In one embodiment, the triptan is sumatriptan and its salts and the NSAID is naproxen and its salts.

In an embodiment of the invention, the multilayer tablet comprises sumatriptan or any of its salts and naproxen or any of its salts, wherein: a) between 10% or more of the naproxen or its salts and up to about 90% thereof is in a first layer of the tablet together with sumatriptan or its salts; and b) the remaining naproxen or its salts is in a second layer of the tablet. In another embodiment, the amount of naproxen in the first layer is more than 10%, and in still another embodiment, 12% or more, 13% or more, 14% or more, 15% or more and 20% or more. The layers may be in a side-by-side, inlay or coated arrangement.

Stated another way, in one embodiment, the present invention is a compressed tablet or a mini-tablet including two layers or more. A first layer includes substantially all of a first active pharmaceutical ingredient (“API”), which is either a triptan or its salt, or an NSAID or its salt, and more than 10% of a second API and a second layer which is generally devoid of the first API and comprises the balance of the second API. These layers may be in a side-by-side relationship, in a coated core where one layer completely surrounds the other, or where one layer is embedded in the other, but at least one surface of each layer is exposed (also “inlay tablets”). The layer which contains a mixture may comprise more than 10% of the second API up to about 90% thereof by weight of the second API. The lower limit can, in some embodiments, be about 11%, 12%, 13%, 14%, 15%, 16%, 17%, 18%, 19%, 20%, 25% or about 30% and the maximum amount can, in some embodiments, be about 80%, 70%, 60%, 50% or about 40%.

One embodiment, the invention is directed to a composition including at least 10% of the sumatriptan succinate that is present (e.g., about 17.85 mg of total dose 119 mg which is 15% of total dose), together with substantially all of the naproxen sodium (e.g., 500 mg), in the first layer and a second layer with the remaining amount of sumatriptan succinate (e.g., 101.15 mg) that is present, wherein both the layers are compressed into a bilayer tablet.

In an embodiment of the invention, sumatriptan and naproxen are presented in a capsule. These can be capsules that include tablets or minitablets, which are layered as described herein.

In yet another embodiment the invention includes granules, powders, or mini-tablets and blends of a triptan or its salts and an NSAID or its salts, filled together into capsule. This is particularly useful for lower strengths, such as 250 mg of naproxen. In one further embodiment, the capsule may be filled with two portions of granules or powders, wherein one portion comprises a triptan and an NSAID and the other portion comprise just the remainder of the triptan.

Further an embodiment the invention includes granules or blends of sumatriptan or its salts and naproxen or its salts together compressed into a monolithic tablet. The monolithic tablet may comprise a blend of two portions of granules or powders, wherein one portion comprises part of a triptan and an NSAID and the other portion comprises the remainder of the triptan. The reverse, where two layers contain the NSAID, is also contemplated for each.

For each of the various embodiments, while any triptan and NSAID may be used, sumatriptan and naproxen are described. And where salts are described, sumatriptan in the form of its succinate salt and naproxen in the form of its sodium salt are described.

Further embodiments the invention includes stable pharmaceutical compositions comprising sumatriptan or its salts and naproxen or its salts, wherein sumatriptan is in the form of its succinate salt and naproxen is in the form of its sodium salt.

In other embodiments, there is provided an inlay tablet comprising: an inner tablet and an outer tablet which encompass all but at least a portion of one face of the inner tablet, the inner tablet comprising a first active pharmaceutical ingredient which is a triptan or a nonsteroidal anti-inflammatory drug, and the outer tablet comprising a second active pharmaceutical ingredient which is a triptan or a nonsteroidal anti-inflammatory drug, and wherein the first and the second active pharmaceutical ingredients are not both triptans or both nonsteroidal anti-inflammatory drugs; a compressed layered tablet or mini-tablet comprising: a first layer comprising least one triptan and at least one nonsteroidal anti-inflammatory drug, wherein the first layer comprises substantially all of one of the triptan or the nonsteroidal anti-inflammatory drug and from about 13% to about 85% by weight of the other, and a second layer which includes the balance of the triptan or the nonsteroidal anti-inflammatory drug present in the tablet or mini-tablet; and a compressed layered tablet or mini-tablet comprising: naproxen sodium and sumatriptan succinate wherein a first layer comprises substantially all of the naproxen sodium present in the tablet or mini-tablet and about 13% by weight of the sumatriptan succinate present therein, and a second layer which includes the balance of the sumatriptan succinate, the layers being arranged side-by-side.

In an embodiment the invention includes processes to prepare compositions.

Further embodiments include methods of using pharmaceutical compositions of the present invention.

One embodiment of the invention is an inlay tablet comprising: an inner tablet and an outer tablet which encompass all but at least a portion of one face of the inner tablet, the inner tablet comprising a first active pharmaceutical ingredient which is a triptan or a nonsteroidal anti-inflammatory drug, and the outer tablet comprising a second active pharmaceutical ingredient which is a triptan or a nonsteroidal anti-inflammatory drug, and wherein the first and the second active pharmaceutical ingredients are not both triptans or both nonsteroidal anti-inflammatory drugs.

In another embodiment, the invention comprises a compressed layered tablet comprising: a first layer comprising substantially all of a first active pharmaceutical ingredient found in the tablet and at least 10%, but less than 90% of the total amount of a second active pharmaceutical ingredient found in the tablet, with the balance of the second active pharmaceutical ingredient disposed in a second layer, the first and the second layers being arranged side-by-side, and wherein the first and second active pharmaceutical ingredients are selected from the group consisting of triptans and nonsteroidal anti-inflammatory drugs, and further when the first active pharmaceutical ingredient is a triptan, the second active pharmaceutical ingredient is a nonsteroidal anti-inflammatory drug and when the first active pharmaceutical ingredient is a nonsteroidal anti-inflammatory drug, the second active pharmaceutical ingredient is a triptan. The tablet may be a mini-tablet, the triptan may be sumitriptan and its salts, and the NSAID may be naproxen and its salts.

In still another embodiment, there is provided a compressed layered tablet or mini-tablet comprising: naproxen sodium and sumatriptan succinate, wherein a first layer comprises substantially all of the naproxen sodium present in the tablet or mini-tablet and about 13% by weight of the sumatriptan succinate found therein, and a second layer which includes the balance of the sumatriptan succinate, the layers being arranged side-by-side.

### DETAILED DESCRIPTION

While the specification concludes with claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

The art often discusses separate dosage forms or dosage forms where all of a triptan and all of an NSAID are mixed together in some sort of admixture. This can be a mixture of separately granulated APIs or a simple mixture, usually with excipients. Where layered dosage forms are disclosed according to the '183 patent, the layers generally are discrete with substantially all of one API in one layer and all of the other API in another layer. This art suggests that the first, an admixture, is undesired because the NSAID can interfere with absorbance of the triptan. See '183 patent, column 3, lines 45-67. But even that is not sufficient. Even a tablet made of a core of a naproxen salt, surrounded completely by a layer of a sumatriptan salt, was found to be unacceptable. See id. at Tables 6-10 and 12.

Yet, it has been discovered that a multilayered dosage form can in fact be constructed wherein at least one layer includes all of one API and more than 10% of the other and the second layer includes the balance of the second API. These formulations are sufficiently stable and have sufficient release characteristics to be acceptable for the treatment of various conditions. In one particular embodiment, where the APIs are sumatriptan and naproxen, and salts thereof, it is believed that a compressed tablet of one of the APIs and 10% or more of the other, with the balance being in the second layer is at least bioequivalent to the TREXIMET tablets.

This approach has several advantages. For one thing, instead of fearing the effect of, for example, naproxen, on the release rate of, for example, sumatriptan, one can harness the rate release effect to help tailor the desired release rate of sumatriptan or another triptan as appropriate. This may provide considerable advancements in terms of the design of the dosage form providing a release profile for a triptan wherein some is released from one layer at one rate and released from a second layer at a second rate. Using such physical means for accomplishing these goals can reduce the need for certain types of excipients or technique which may themselves impart costs or concerns about chemical instability or incompatibility.

In addition, in conventional side-by-side layered tablets where the two tablets are formed together in a conventional methodology, considerable blending occurring at the interface. The '183 patent would require extra care and vigilance in ensuring that, as a result of such blending, less than 10%, preferably less than 5%, of one active is mixed with the other. Indeed, the '183 patent discusses the possibility of producing the tablets separately and/or with a barrier layer to physically prevent any intermingling. According to the present invention, it has been discovered that such steps are unnecessary, saving considerably in terms of formulation efficiency and cost.

Triptans may include, without limitation, sumatriptan, eletriptan, rizatriptan, frovatriptan, almotriptan, zolmitriptan, and naratriptan, including pharmaceutically acceptable salts and esters thereof. Sumatriptan, including pharmaceutically acceptable salts and esters thereof, is used in one particular embodiment and may be in the form of sumatriptan free base or its pharmaceutically acceptable salts, esters or isomers, in any polymorphic or amorphous form or combinations thereof. Sumatriptan is used in the invention in the range of 1 mg to 300 mg, or 25 mg to 100 mg, per dosage unit based on the weight of the contained free base equivalent.

NSAIDs may include, without limitation, flurbiprofen, ketoprofen, naproxen, oxaprozin, etodolac, indomethacin, ketorolac, nabumetone, mefanamic acid, piroxicam, lornoxicam, and meloxicam, including pharmaceutically acceptable salts or esters thereof. Another group of NSAIDs that can be used is the cyclooxygenase-2 (COX-2) inhibitors. Members of this group include rofecoxib, celecoxib, valdecoxib, etoricoxib, JTE-522, L-745,337, NS398, and pharmaceutically acceptable salts thereof. Naproxen is used in one particular embodiment and may be in the form of naproxen free base or any of its pharmaceutically acceptable salts or isomers, in any polymorphic or amorphous form or combinations thereof. Naproxen used in the invention is in the range of 250 mg to 550 mg, per dosage unit based on the weight of the contained free base equivalent.

A recitation of “triptan” or “NSAID” herein, either generally or as a specific drug compound, will be used to include both the drug in its free form, and in the form of any substances that provide the drug to the body, following ingestion of a pharmaceutical composition. Such substances can be salts, esters, or other derivatives of the drug.

Inlay tablets according to the present invention are tablets wherein a first tablet (“inner tablet”) is positioned within a comparatively larger “outer” tablet in such a way that at least one surface, face, or part, of the first tablet is not in contact with a surface of the other, relatively larger, outer tablet. Note that “smaller” and “larger” in this context generally relate to comparative surface area. The inner tablet may be larger in terms of volume, weight or dose. However, it is at least partially and generally substantially surrounded by the “outer” tablet.

In an embodiment of the invention, fixed dose pharmaceutical compositions comprising sumatriptan and naproxen are presented as inlay tablets, wherein an outer tablet comprises either sumatriptan or naproxen, and an inner tablet comprises sumatriptan when an outer tablet comprises naproxen, or an inner tablet comprises naproxen when the outer tablet comprises sumatriptan. In a further embodiment, the naproxen is present as the sodium salt and the sumatriptan is present as the succinate salt. In still another embodiment, either the inner or the outer tablet includes a mixture of both naproxen and its salts and sumatriptan and its salts, while the other layer comprises only one of these two APIs. In a further embodiment, the amount of one of these APIs is substantially all of that API present in the dosage form. However, the second API is present in the mixed API layer in an amount of at least 10% by weight of the total amount of that API in the overall dosage form. It can be present in an amount of about 11%, 12%, 13%, 14%, 15%, 16%, 17%, 18%, 19%, 20%, 25%, and 30%, or more, up to about 90%.

In an embodiment of the invention, a multilayer tablet comprises sumatriptan and naproxen wherein: a) 10% or more of the sumatriptan is in a first layer of the tablet along with substantially all of the naproxen; and b) the remaining sumatriptan is in a second layer of the tablet. These may be side-by-side layered tablets and coated tablets where one layer completely surrounds the other, as well as inlay tablets as described.

In an embodiment of the invention, a multilayer tablet comprises sumatriptan succinate and naproxen sodium wherein: a) 10% or more of the sumatriptan succinate is in a first layer of the tablet along with substantially all of the naproxen sodium; and b) the remaining sumatriptan succinate is in a second layer of the tablet. These may be side-by-side layered tablets and coated tablets where one layer completely surrounds the other, as well as inlay tablets as just described.

In an embodiment of the invention, a multilayer tablet comprises sumatriptan and naproxen wherein: a) 10% or more of the naproxen is in a first layer of the tablet along with substantially all of the sumatriptan; and b) the remaining naproxen is in a second layer of the tablet. These may be side-by-side layered tablets and coated tablets where one layer completely surrounds the other, as well as inlay tablets as just described.

In an embodiment of the invention, a multilayer tablet comprises sumatriptan succinate and naproxen sodium wherein: a) 10% or more of the naproxen sodium is in a first layer of the tablet along with substantially all of the sumatriptan succinate; and b) the remaining naproxen sodium is in a second layer of the tablet. These may be side-by-side layered tablets and coated tablets where one layer completely surrounds the other, as well as inlay tablets as just described. All of the foregoing may also be mini-tablets which can be filled into a capsule. And all of the tablets and mini-tablets may be coated with other materials including rate controlling materials.

In an embodiment of the invention, wherein part of the sumatriptan or naproxen is placed in a layer comprising the other drug, that part of the sumatriptan or naproxen can be presented as uncoated or coated granules, particles, pellets or beads.

In an embodiment of the invention, sumatriptan and naproxen are presented in a capsule. In an embodiment of the invention wherein the composition is presented as capsules, capsules comprise sumatriptan and naproxen, wherein either one or both of sumatriptan and naproxen are presented as a powder, granules, coated granules, beads, coated beads, pellets, coated pellets, tablets, coated tablets, mini-tablets, coated mini-tablets, or as two separate smaller capsules, wherein each capsule is filled with sumatriptan and naproxen.

In one of the embodiments the invention includes granules or blends of sumatriptan succinate and naproxen sodium, filled together into empty hard gelatin capsules.

Further embodiments the invention includes stable pharmaceutical compositions comprising sumatriptan or its salts and naproxen or its salts, wherein sumatriptan is in the form of its succinate salt and naproxen is in the form of its sodium salt.

The following are some potential impurities of sumatriptan succinate which could be formed in the formulation during processing or during storage conditions:

1) Impurity A, chemically known as 3-[2(Dimethylamino)ethyl]-2-[[3-[2-(dimethylamino)ethyl]-1H indol-5-yl]methyl]-1H-indol-5-yl]-N-Methyl methane sulphonamide, and represented by structure IV.

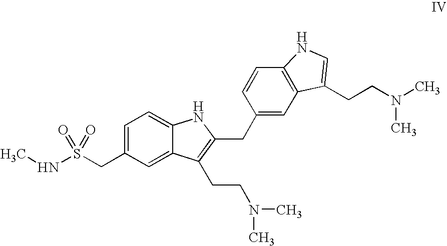

2) Impurity B, chemically known as N-Methyl[3-[2-(methyl amino)ethyl]-1H-indol-5-yl]methane sulphonamide, and represented by structure V.

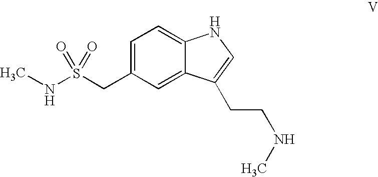

3) Impurity C, chemically known as [3-[2-(dimethylamino)ethyl]-1-(hydroxyl methyl)-1H-indol-5-yl]-N-methane sulphonamide, and represented by structure VI.

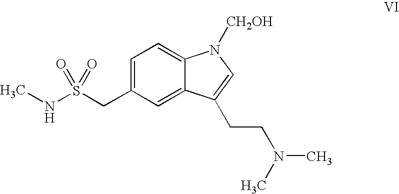

4) Impurity D, chemically known as N,N-Dimethyl-2-[5-[(methyl sulphamoyl)methyl]-1H indol -3-yl]ethanamine N-oxide, and represented by structure VII.

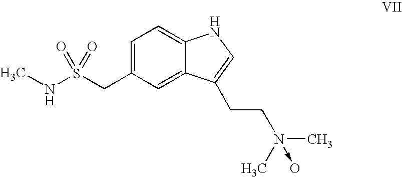

Some potential impurities of naproxen sodium are as below:

5) Impurity E, chemically known as 2-Acetyl-methoxy naphthalene, and represented by structure VIII.

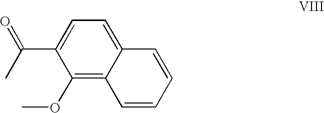

6) Impurity F, chemically known as 2-methyl-6-methoxy-a-methyl-2-napthaleneacetate, and represented by structure IX.

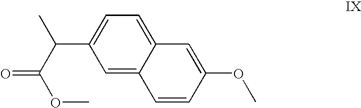

7) Impurity G, chemically known as 3-isopropyl-6-methoxy-a-methyl-2-napthaleneacetate, and represented by structure X.

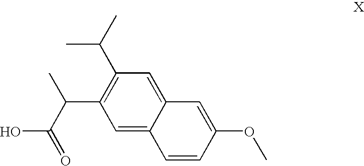

8) Impurity H, chemically known as 1-(6-methoxy-2-naphthyl)ethylalcohol, and represented by structure XI.

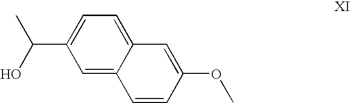

9) Impurity I, chemically known as n-octyl d-glucamine, and represented by structure XII.

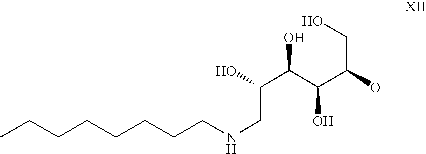

In yet another embodiment the invention includes granules or blends of sumatriptan or its salts and naproxen or its salts, together compressed into multilayered tablets including side-by-side tablets, inlay tablets or tablets in which one layer or tablet surrounds the other. These include mini-tablets. Sumatriptan and naproxen powders, granules, beads, pellets or tablets can be prepared using techniques known to persons skilled in the art. Techniques that can be used include but are not limited to any one or more of direct blending, dry granulation, or wet granulation techniques, extrusion-spherinization, drug layering onto inert materials, direct compression, wet granulation followed by compression techniques, or any other pharmaceutical formulation techniques.

Blends or granules comprising active ingredients may comprise one or more of pharmaceutically acceptable excipients such as but not limited to diluents, disintegrants, binders, lubricants, surfactants, glidants, coloring agents, film formers, plasticizers, opacifying agents, sweeteners, flavoring agents and the like.

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different starches include but are not limited to maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products) and others. Different celluloses that can be used include crystalline cellulose and powdered cellulose. Examples of crystalline cellulose products include but are not limited to CEOLUS™ KG801, Avicel™ PH101, PH102, PH301, PH302 and PHF20, and PH112, microcrystalline cellulose 114, and microcrystaline cellulose 112. Other useful diluents include but are not limited to carmellose, sugar alcohols such as mannitol (Pearlitol™ SD200), sorbitol, and xylitol, calcium carbonate, magnesium carbonate, dibasic calcium phosphate, and tribasic calcium phosphate. The amounts used are consistent with known formulation practice, but generally range from about 5% to about 60%, and in another embodiment, from about 15% to about 40%, by weight of the tablet or layer as appropriate.

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches. The amounts used are consistent with known formulation practice, but generally range from about 0.5 to about 20%, and in another embodiment, from about 2 to about 10%, by weight of the tablet or layer as appropriate.

Various useful binders include but are not limited to hydroxypropylcelluloses, also called HPC (Klucel™-LF, Klucel EXF) and useful in various grades, hydroxypropyl methylcelluloses, also called hypromelloses or HPMC (Methocel™) and useful in various grades, polyvinylpyrrolidones or povidones (such as grades PVP-K25, PVP-K29, PVP-K30, and PVP-K90), Plasdone™ S 630 (copovidone), powdered acacia, gelatin, guar gum, carbomers (Carbopol™), methylcelluloses, polymethacrylates, and starches. The amounts used are consistent with known formulation practice, but generally range from about 0.5 to about 20%, and in another embodiment, from about 1 to about 7.5%, by weight of the tablet or layer as appropriate.

Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof. The amounts used are consistent with known formulation practice, but generally range from about 0.1 to about 10%, and in another embodiment, from about 0.5 to about 5%, by weight of the tablet or layer as appropriate.

Useful surface-active agents include non-ionic, cationic or anionic surface-active agents. Useful non-ionic surface-active agents include ethylene glycol stearates, propylene glycol stearates, diethylene glycol stearates, glycerol stearates, sorbitan esters (SPAN™), polyoxyethylene derivatives of sorbitan esters, or polysorbates (TWEEN™), aliphatic alcohols and PEG ethers, phenol and PEG ethers. Useful cationic surface-active agents include quaternary ammonium salts (e.g. cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk. The amounts used are consistent with known formulation practice, but generally range from about 0.1 to about 10% and in another embodiment, from about 0.5 to about 5%, by weight of the tablet or layer as appropriate.

Various solvents can be used in the processes of preparation of pharmaceutical compositions of the present invention including but not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and any mixtures thereof. Typically, solvents will evaporate during the formulation processes.

Coloring agents can be used to color code the compositions, for example, to indicate the type and dosage of the therapeutic agent therein. Suitable coloring agents include, without limitation, natural and/or artificial compounds such as FD&C coloring agents, natural juice concentrates, pigments such as titanium oxide, silicon dioxide, iron oxides, and zinc oxide, combinations thereof, and the like. The amounts used are consistent with known formulation practice, but generally range from about 0.001 to about 2%, and in another embodiment, from about 0.005 to about 0.1%, by weight of the tablet or layer as appropriate.

One or more glidant materials, which improve the flow properties of powder blends and minimize dosage form weight variation, can be used. Useful glidants include but are not limited to silicone dioxide, talc and combinations thereof. The amounts used are consistent with known formulation practice, but generally range from about 0.5 to about 10%, and in another embodiment, from about 1 to about 5%, by weight of the tablet or layer as appropriate.

Film-forming polymers that are useful in the invention include but are not limited to cellulose derivatives such as hydroxypropyl celluloses, hydroxyethyl celluloses, hydroxypropyl methylcelluloses, methacrylic acid copolymers, lactose, starch derivatives, and the like. The amounts used are consistent with known formulation practice, but generally range from about 1 to about 20%, and in another embodiment, from about 1 to about 10%, by weight of the tablet or layer as appropriate.

Various useful plasticizers for film coatings include but are not limited to substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titanium dioxide may also be present in an amount ranging from about 10% to about 20%, based on the total weight of the coating. Film coatings can also contain coloring agents.

Coating can also be accomplished using pre-formulated coating products, which usually require only mixing with a liquid before use. Useful products include those sold by Colorcon as OPADRY™ or OPAGLOSS, for example OPAGLOSS 2 Blue (97W80859) and others. The amounts used are consistent with known formulation practice, but generally range from about 1 to about 15%, and in another embodiment, from about 1 to about 10%, by weight of the tablet or layer as appropriate.

In an aspect the invention includes processes to prepare pharmaceutical compositions, wherein a specific embodiment of a process comprises:

1) sifting drug and excipients such as diluents, disintegrants, glidants, lubricants etc through a sieve;

2) dry mixing sifted ingredients except glidants and lubricants;

3) optionally, granulating the step 2) materials using a binder solution or dispersion, and subsequently drying and sizing through a sieve;

4) optionally, compacting the step 2) materials into compacts and subsequently milling and sizing through a sieve;

5) blending either step 2) or step 3) or step 4) product with sifted glidant and other excipients, if any;

6) adding sifted lubricant to step 5) materials and blending;

7) filling the step 6) product into capsules or compressing into tablets.

The formulations prepared are further packaged using appropriate packaging materials such as containers, including closures, composed of polyethylene (high density polyethylene or low density polyethylene), and/or polypropylene, and/or glass, stainless steel bottles, etc. Also useful are various blisters or strips composed of aluminum or high-density polypropylene, or polyvinyl chloride, or polyvinyl chloride (PVC) coated with polyvinylidene dichloride (PVDC), generally termed PVC/PVDC. Different grades of PVC/PVDC are available as PVC/PVDC 40 gsm, PVC/PVDC 60 gsm, PVC/PVDC 90 gsm, etc., where “gsm” indicates the grams of PVDC coating per square meter of PVC film. For stability various moisture adsorbents like silica gel (Sorb-IT® supplied by Multisorb Technologies is a prepackaged sachet-style packet of silica gel commonly used in the packaging), molecular sieves, etc. and oxygen absorbers such as Ageless® (manufactured by Mitsubishi Gas Chemical), or Stabilox® (manufactured by Multisorb Technologies) are included in packaged products.

Sometimes a pharmaceutical coil may be used during packaging to provide a safe, nontoxic, aesthetically pleasing, easy to use, traditional filler. It ensures freshness and stability in shipping and storing. An example polyester coil (supplied by Carolina Inc, Pharmaceutical coil) is white, odorless, soft, low moisture, resilient material comprised of 100% polyester fibers.

The pharmaceutical dosage forms of the present invention are intended for oral administration to a patient in need thereof.

In an embodiment, the invention relates to analytical methods for analysis of sumatriptan- and naproxen-related impurities using high performance liquid chromatography (HPLC), wherein a method comprises:

For Sumatriptan-Related Impurities:

Mobile phase A: 1.36 g of potassium dihydrogen phosphate (anhydrous) and 1.5 mL of dibutylamine are added to 1000 mL of water, and pH is adjusted to 3.0 with orthophosphoric acid. The solution is filtered through 0.45 μm hydrophilic membrane filter and degassed in a sonicator for about 10 minutes.

Mobile phase B: Water, methanol and acetonitrile are mixed in the volume ratio of 200:150:650, respectively, followed by degassing in a sonicator for 10 minutes

Diluent: Mobile phase A, methanol and acetontrile are mixed in the volume ratio of 500:250:250, respectively.

Chromatographic System:

a) Liquid chromatograph is equipped with a 225 nm UV detector.

b) 250 mm×4.6 mm, 5 μm, Waters Spherisob ODS-1 or equivalent column.

c) Column temperature: 25° C.

d) Flow rate: 1 mL per minute.

e) Injection volume: 10 μL.

f) Run time: 70 minutes.

Mobile Phase Gradient Program:

| Time | % of Mobile | % of Mobile |
|---|---|---|
| (minutes) | Phase A | Phase B |
| 0 | 95 | 5 |
| 5 | 95 | 5 |
| 35 | 90 | 10 |
| 50 | 50 | 50 |
| 55 | 40 | 60 |
| 57 | — | 100 |
| 60 | — | 100 |
| 62 | 95 | 5 |
| 70 | 95 | 5 |

Preparation of Test Sample:

Accurately weigh a portion of crushed tablet powder equivalent to 100 mg of sumatriptan succinate into a 100 mL volumetric flask. To this, 70 mL of diluent is added and sonicated for 20 minutes with intermediate shaking. The volume is made up with diluent, followed by centrifuging a portion of the solution in a centrifuge tube with a cap at 4000 rpm for about 10 minutes. Finally, the solution is filtered through a 0.45 μm hydrophilic membrane filter.

For Naproxen Sodium-Related Impurities:

Mobile Phase: Water, acetonitrile and glacial acetic acid are mixed in the volume ratio of 50:50:1 and filtered through 0.45 μm nylon 66 membrane filter, followed by degassing in a sonicator for about 10 minutes.

Diluent: 900 mL of acetonitrile is mixed with 100 mL of milli-Q water, followed by degassing in a sonicator for about 10 minutes.

Sample Preparation:

Accurately weighed amount of the crushed tablet powder equivalent to 250 mg of naproxen sodium is placed in a 100 mL volumetric flask. To this, 70 mL of diluent is added and sonicated for about 20 minutes, then diluted to volume with diluent. Centrifuge a portion of the above solution in a centrifuge tube with cap at 4000 rpm for 10 minutes.

Chromatographic System:

a) Liquid chromatograph is equipped with a 260 nm UV detector.

b) Column: 250 mm×4.6 mm, Symmetryshield, RP18, 5 μm column or equivalent.

c) Column temperature: Ambient.

d) Flow rate: 1 mL per minute.

e) Injection volume: 10 μL.

f) Run time: 50 minutes for blank and 15 minutes for standard.

Representative relative retention times (RRT), where sumatriptan or naproxen equals 1, limits of detection (LOD), and limits of quantification (LOQ) of various drug-related impurities are tabulated below.

| Drug | Impurity | RRT | LOD | LOQ |
|---|---|---|---|---|
| Sumatriptan | Impurity A | 2.10 | 0.005 | 0.017 |
| | Impurity B | 0.78 | 0.005 | 0.015 |
| | Impurity C | 1.15 | 0.008 | 0.020 |
| | Impurity D | 1.57 | 0.008 | 0.022 |
| Naproxen | Impurity E | 1.32 | 0.00033 | 0.001 |
| | Impurity F | 2.24 | 0.005 | 0.017 |
| | Impurity G | 4.64 | 0.011 | 0.032 |
| | Impurity H | 0.84 | 0.001 | 0.006 |

Pharmaceutical dosage forms can be subjected to in vitro dissolution evaluations according to Test 711 “Dissolution” in United States Pharmacopoeia 29, United States Pharmacopeial Convention, Inc., Rockville, Md., 2005 (“USP”), to determine the rate at which the drug substance is released from the dosage forms, and the content of drug substance can conveniently be determined in solutions using techniques such as high performance liquid chromatography (HPLC).

Certain specific aspects and embodiments of the invention will be explained in more detail with reference to the following examples, being provided only for purposes of illustration, and it is to be understood that the present invention is not to be limited thereto.

### EXAMPLE 1

### Inlay Tablets Containing Sumatriptan Succinate 119 mg and Naproxen Sodium 500 mg

| | |
|---|---|
| Ingredient | Percent |

Inner Smaller Tablet

| | |
|---|---|
| Sumatriptan succinate | 10 |
| Lactose monohydrate | 10 |
| Water* | q.s. |
| Anhydrous lactose | 20.1 |
| Microcrystalline cellulose | 2.2 |
| Croscarmellose sodium | 0.4 |
| Magnesium stearate | 0.2 |

Outer Larger Tablet

| | |
|---|---|
| Naproxen sodium | 42.2 |
| Microcrystalline cellulose | 4.5 |
| Povidone | 2 |
| Water* | q.s. |
| Microcrystalline cellulose | 4.5 |
| Croscarmellose sodium | 1.1 |
| Talc | 2.3 |
| Magnesium stearate | 0.4 |

*Evaporates during processing.

Manufacturing Process:

A. Inner Tablet.

1) Separately sift sumatriptan succinate, lactose monohydrate, anhydrous lactose, microcrystalline cellulose, and croscarmellose sodium through an ASTM #40 mesh sieve, and sift magnesium stearate through an ASTM #80 mesh sieve.

2) Place sumatriptan succinate and lactose monohydrate into a granulator and dry mix.

3) Granulate step 2) using water.

4) Dry the granules until the loss on drying is not more than 2% w/w at 60° C.

5) Sift the dried granules through an ASTM #20 mesh sieve.

6) Blend the granules of step 5) with anhydrous lactose, microcrystalline cellulose, and croscarmellose sodium in a double cone blender for about 10 minutes.

7) Blend step 6) materials with magnesium stearate for about 5 minutes.

8) Compress the lubricated blend into tablets.

B. Outer Tablet Blend.

1) Co-sift naproxen sodium and microcrystalline cellulose (first quantity) through an ASTM #40 mesh sieve.

2) Place step 1) materials into a granulator and dry mix.

3) Dissolve povidone in water to prepare a binder solution and granulate step 2), then dry until the loss on drying is not more than 2% w/w at 60° C.

4) Sift the granules of step 3) through an ASTM #20 mesh sieve.

5) Co-sift microcrystalline cellulose (second quantity), croscarmellose sodium through an ASTM #40 mesh sieve.

6) Blend the granules of step 4) with step 5) materials in a double cone blender for about 10 minutes.

7) Sift talc and magnesium stearate through an ASTM #80 mesh sieve.

8) Blend step 6) with step 7) materials.

C. Inlay Tablet.

1) Compress inner tablets together with the outer tablet blend into inlay tablets.

### EXAMPLE 2

### Bilayer Side-by-Side Tablets Containing Sumatriptan Succinate 119 mg and Naproxen Sodium 500 mg; about 15% of Sumatriptan Succinate is in the Naproxen Layer

| | |
|---|---|
| Ingredient | Percent |

Layer 1

| | |
|---|---|
| Sumatriptan succinate | 8.5 |
| Lactose monohydrate | 10 |
| Water* | q.s. |
| Anhydrous lactose | 20.1 |
| Microcrystalline cellulose | 2.2 |
| Croscarmellose sodium | 0.4 |
| Magnesium stearate | 0.2 |

Layer 2

| | |
|---|---|
| Naproxen sodium | 42.2 |
| Microcrystalline cellulose | 4.5 |
| Povidone | 2 |
| Water* | q.s. |
| Sumatriptan succinate | 1.5 |
| Microcrystalline cellulose | 4.5 |
| Croscarmellose sodium | 1.1 |
| Talc | 2.3 |
| Magnesium stearate | 0.4 |

*Evaporates during processing.

Manufacturing Process:

Layer 1.

1) Sift sumatriptan succinate, lactose monohydrate, anhydrous lactose, microcrystalline cellulose, croscarmellose sodium through an ASTM #40 mesh sieve separately, and sift magnesium stearate through an ASTM #80 mesh sieve.

2) Place sumatriptan succinate and lactose monohydrate of step 1) into a granulator and dry mix.

3) Granulate step 2) materials using water.

4) Dry the granules until loss on drying is not more than 2% at 60° C.

5) Sift the granules of step 4) through an ASTM #20 mesh sieve.

6) Blend anhydrous lactose, microcrystalline cellulose, and croscarmellose sodium with granules of step 5) in a double cone blender for about 10 minutes.

7) Blend step 6) with magnesium stearate for about 5 minutes.

Layer 2.

1) Co-sift naproxen sodium and microcrystalline cellulose (first quantity) through an ASTM #40 mesh sieve.

2) Place step 1) materials into a granulator and dry mix.

3) Dissolve povidone in water to form a binder solution and granulate step 2) materials using the binder solution.

4) Dry the granules until loss on drying is not more than 2% w/w at 60° C.

5) Sift the granules through an ASTM #20 mesh sieve.

6) Co-sift sumatriptan succinate, microcrystalline cellulose (second quantity), and croscarmellose sodium through an ASTM #40 mesh sieve.

7) Blend the granules of step 5) with step 6) materials in a double cone blender for about 10 minutes.

8) Sift talc and magnesium stearate through an ASTM #80 mesh sieve.

9) Blend step 7) materials with step 8).

C. Bilayer Tablet.

1) Compress Layer 1 blend and Layer 2 blend together to form bilayer tablets.

### EXAMPLE 3

### Bilayer Tablets Containing Sumatriptan Succinate 119 mg and Naproxen Sodium 500 mg; about 15% of Sumatriptan Succinate is in a Naproxen Layer

| | |
|---|---|
| Ingredient | Percent |

Layer 1 Intragranular

| | |
|---|---|
| Sumatriptan succinate | 8.87 |
| Dibasic calcium phosphate dihydrate | 10.44 |
| Croscarmellose sodium | 1.75 |
| FD&C blue lake blend | 0.01 |
| Water* | q.s. |

Layer 1 Extragranular

| | |
|---|---|
| Microcrystalline cellulose | 8.57 |
| Sodium bicarbonate | 3.51 |
| Magnesium stearate | 0.35 |
| Layer 1 Total | 33.5 |

Layer 2 Intragranular

| | |
|---|---|
| Naproxen sodium | 43.86 |
| Microcrystalline cellulose | 4.74 |
| Povidone | 2.37 |
| Water* | q.s. |
| Sumatriptan succinate | 1.57 |
| Povidone | 0.08 |
| Water* | q.s. |

Layer 2 Extragranular

| | |
|---|---|
| Microcrystalline cellulose | 3.46 |
| Crospovidine | 2.72 |
| Talc | 2.37 |
| Magnesium stearate | 0.59 |
| Layer 2 Total | 61.76 |
| Total for compressed tablet | 95.26 |

Coating

| | |
|---|---|
| Opagloss II Blue (97W80859) | 4.74 |
| Total for coated tablet | 100 |

*Evaporates during processing.

Manufacturing Process:

Layer 1.

1) Separately sift sumatriptan succinate, dibasic calcium phosphate dihydrate and croscarmellose sodium through an ASTM #40 mesh sieve, and mix for 5 minutes in a rapid mixer granulator (RMG).

2) Disperse FD&C lake blend in water.

3) Granulate the mixture of step 1) with the step 2) dispersion.

4) Dry the granules of step 3) in a fluid bed dryer (FBD) to obtain a loss on drying not more than 2% at 60° C.

5) Sift the dried granules of step 4) through an ASTM #30 mesh sieve.

6) Sift microcrystalline cellulose and sodium bicarbonate through an ASTM #40 mesh sieve separately.

7) Blend the granules of step 5) and sifted materials of step 6) in a double cone blender for about 5 minutes.

8) Sift magnesium stearate through an ASTM #60 mesh sieve.

9) Blend the mixture of step 7) with magnesium stearate of step 8) for about 5 minutes.

Layer 2

1) Co-sift naproxen sodium and microcrystalline cellulose through an ASTM #30 mesh sieve.

2) Dissolve povidone in water

3) Spray the solution of step 2) onto the blend of step 1) in a fluid bed processor fitted with top spray, to prepare granules.

4) Dissolve sumatriptan succinate in water together with povidone.

5) Spray the solution of step 4) onto the granules of step 3).

6) Dry the granules of step 5) in the FBD to obtain a loss on drying not more than 2% at 70° C.

7) Sift microcrystalline cellulose and crospovidone through an ASTM #40 mesh sieve separately.

8) Blend the granules of step 6) and sifted materials of step 7) in a double cone blender for about 5 minutes.

9) Sift magnesium stearate and talc through an ASTM #60 mesh sieve.

10) Blend the mixture of step 8) with sifted materials of step 9) for about 5 minutes.

Bilayer Tablets

1) Compress the Layer 1 blend and Layer 2 blend together to form bilayer tablets.

2) Coat the tablets of step 1) with a dispersion of Opagloss II Blue.

### EXAMPLE 4

### Bilayer Tablets Containing Sumatriptan Succinate 119 mg and Naproxen Sodium 500 mg;n about 15% of Naproxen Sodium is in a Sumatriptan Layer

| | |
|---|---|
| Ingredient | Percent |

Layer 1

| | |
|---|---|
| Sumatriptan succinate | 10 |
| Lactose monohydrate | 10 |
| Water* | q.s. |
| Naproxen sodium | 6.3 |
| Anhydrous lactose | 20.1 |
| Microcrystalline cellulose | 2.2 |
| Croscarmellose sodium | 0.4 |
| Magnesium stearate | 0.2 |

Layer 2

| | |
|---|---|
| Naproxen sodium | 35.9 |
| Microcrystalline cellulose | 4.5 |
| Povidone | 2 |
| Water* | q.s. |
| Microcrystalline cellulose | 4.5 |
| Croscarmellose sodium | 1.1 |
| Talc | 2.3 |
| Magnesium stearate | 0.4 |

*Evaporates during processing.

Manufacturing process: similar to that of Example 2, except include naproxen sodium in step 6) with extragranular materials of Layer 1.

### EXAMPLE 5

### Capsules Containing Sumatriptan Succinate 119 mg and Naproxen Sodium 250 mg

| | |
|---|---|
| Ingredient | Percent |

Blend 1

| | |
|---|---|
| Sumatriptan succinate | 23.8 |
| Lactose monohydrate | 9 |
| Water* | q.s. |
| Anhydrous lactose | 9 |
| Microcrystalline cellulose | 1.1 |
| Croscarmellose sodium | 0.5 |
| Magnesium stearate | 0.2 |

Blend 2

| | |
|---|---|
| Naproxen sodium | 50 |
| Microcrystalline cellulose | 4 |
| Povidone | 0.2 |
| Water* | q.s. |
| Microcrystalline cellulose | 1 |
| Croscarmellose sodium | 0.5 |
| Talc | 0.5 |
| Magnesium stearate | 0.2 |

*Evaporates during processing.

Manufacturing Process:

1) Prepare Blend 1 according to the process for the inner tablet of Example 1 and compress into mini-tablet 1.

2) Prepare Blend 2 according to the process for the outer tablet blend of Example 1 and compress into mini-tablet 2.

3) Fill a mini-tablet 1 and a mini-tablet 2 into an empty hard gelatin capsule shell.

### EXAMPLE 6

### Bilayer Tablet Comprising Sumatriptan 119 mg and Naproxen 500 mg

| | |
|---|---|
| Ingredient | Percent |

Blend 1

| | |
|---|---|
| Sumatriptan succinate | 10 |
| Naproxen sodium | 6.3 |
| Polymer‡ | 0.4 |
| Lactose monohydrate | 10 |
| Methanol | q.s. |
| Water* | q.s. |
| Anhydrous lactose | 20 |
| Microcrystalline cellulose | 2.2 |
| Croscarmellose sodium | 0.4 |
| Magnesium stearate | 0.2 |

Blend 2

| | |
|---|---|
| Naproxen sodium | 35.7 |
| Microcrystalline cellulose | 4.5 |
| Povidone K-30 | 2 |
| Water* | q.s. |
| Microcrystalline cellulose | 4.5 |
| Croscarmellose sodium | 1.1 |
| Talc | 2.3 |
| Magnesium stearate | 0.4 |

*Evaporates during processing.
‡Polymer is any one or more of Eudragit ™ EPO, HPMC, povidone, and Eudragit RD 100.

Manufacturing Process:

Blend 1.

1) Dissolve naproxen sodium and polymer in a methanol and water mixture.

2) Sift sumatriptan succinate and lactose monohydrate through an ASTM #40 mesh sieve. Dry mix for about 5 minutes.

3) Granulate step 2) materials using step 1) solution.

4) Dry the granules in a dryer until loss on drying is not more than 2% at 60° C.

5) Sift the granules of step 4) through an ASTM #20 mesh sieve.

6) Blend anhydrous lactose, microcrystalline cellulose, and croscarmellose sodium with granules of step 5) in a double cone blender for about 10 minutes.

7) Blend the step 6) mixture with magnesium stearate for about 5 minutes.

Blend 2. Prepare blend 2 according to the naproxen layer process in Example 2.

Compression. Blend 1 and blend 2 are compressed together as a monolithic tablet or as a bilayer tablet.

### EXAMPLE 7

### Bilayer Tablet Comprising Sumatriptan 119 mg and Naproxen 500 mg; Approximately 15% Sumatriptan in the Naproxen Layer

| | | |
|---|---|---|
| Ingredient | mg/Tablet | Percent |

Layer 1

| | | |
|---|---|---|
| Naproxen Sodium | 500 | 44.76 |
| Avicel PH101 (MCC) | 54 | 4.8 |
| Povidone (PVPK90) | 27 | 2.41 |
| Water* | q.s. | q.s. |
| Sumatriptan Succinate | 17.85 | 1.6 |
| Povidone (PVPK90) | 1 | 0.09 |
| Water* | q.s. | q.s. |
| Microcrystalline cellulose | 39.4 | 3.53 |
| (Avicel PH102) | | |
| Crospovidone | 31 | 2.76 |
| Talc | 27 | 2.42 |
| Magnesium stearate | 6.75 | 0.6 |
| Layer 1 Total | | 62.97 |

Layer 2

| | | |
|---|---|---|
| Sumatriptan succinate | 101.15 | 9.06 |
| Dibasic calcium phosphate dihydrate | 119 | 10.65 |
| Croscarmellose sodium | 20 | 1.8 |
| Lake Blend Blue (LB 332) | 0.13 | 0.01 |
| Water* | q.s. | q.s. |
| Microcrystalline cellulose | 97.72 | 8.75 |
| (Avicel PH 102) | | |
| Sodium bicarbonate | 40 | 3.6 |
| Magnesium stearate | 4 | 0.4 |
| Layer 2 Total | 382 | 34.27 |
| Total for compressed tablet | 1086 | |

Coating

| | | |
|---|---|---|
| Opagloss 2 Blue (97W80859)@ | 31 | 2.76 |
| Water* | q.s. | q.s. |
| Total for coated tablet | 1117 | 100 |

*Evaporates during processing.
@ Opagloss 2 Blue (97W80859) contains maltodextrin, titanium dioxide, dextrose monohydrate, sodium carboxymethyl cellulose, lecithin, FDC blue # 2, and is supplied by Colorcon Asia Private Limited.

Manufacturing Process:

Blend for Layer 1.

1) Naproxen sodium and Avicel PH 101 are sifted through an ASTM #24 mesh sieve and transferred to a fluid bed processor.

2) The material of step 1) is pre-warmed for about 5-10 minutes.

3) PVP K 90 is dissolved in water to prepare a binder solution.

4) Binder solution of step 3) is sprayed onto step 2) materials.

5) Wet granules from step 4) are partially dried at an inlet temperature of about 65±10° C., until loss on drying at 65±5° C. less than about 15% w/w is achieved.

6) Sumatriptan succinate and povidone are dissolved in water and this solution is sprayed on above step 6) partially dried granules. The granules are dried at 65±10° C., until loss on drying less than about 2% w/w is achieved.

7) The dried granules are sifted through an ASTM #24 mesh sieve. Oversize granules are milled through a 1.5 mm screen and again sifted through the ASTM #24 mesh sieve.

8) Avicel PH 102 and crospovidone are sifted through an ASTM #40 mesh sieve and added to step 7) granules, followed by blending for about 5 minutes.

9) Talc and magnesium stearate are sifted through an ASTM #60 mesh sieve and added to step 8) blend, followed by blending for about 5 minutes.

Blend for Layer 2.

1) Sumatriptan succinate, dibasic calcium phosphate, and croscarmellose sodium are sifted through an ASTM #30 mesh sieve.

2) Lake blend blue is dispersed in water.

3) Step 1) materials are placed into a granulator and dry mixed for about 5 minutes.

4) Step 3) materials are granulated using step 2) dispersion.

5) The wet granules are dried at 65±10° C. until loss on drying less than abut 2% w/w is achieved.

6) The dried granules are sifted through an ASTM #30 mesh sieve. Oversize granules are milled through a 1 mm screen and again sifted through the ASTM #30 mesh sieve.

7) Avicel PH102 and sodium bicarbonate are sifted through an ASTM #40 mesh sieve and added to step 6) granules, followed by blending for about 5 minutes.

8) Magnesium stearate is sifted through an ASTM #40 mesh sieve and added to step 7) blend, followed by blending for about 5 minutes.

Compression

1) Layer 1 and layer 2 blends are compressed into bilayer tablets.

Coating

1) Opagloss 2 Blue is dispersed in water and stirred for about 45 minutes.

2) The above compressed tablets are coated with dispersion from step 1) until about 3% weight gain is achieved.

Stability Data:

Tablets prepared as above are packed in closed high density polyethylene containers with a 1 g Sorb-it silica gel pouch and polyester coil, and stored for 3 months at accelerated stability conditions of 40° C. and 75% RH. Samples are analyzed for water content by the Karl Fischer method, dissolution, and for impurities by HPLC. The data are tabulated below. Dissolution conditions: 900 ml of pH 6.8 phosphate buffer in USP type I apparatus with sinkers, at 50 rpm stirring.

| | | | |
|---|---|---|---|
| Parameter | Initial | 1 Month | 3 Months |

Impurities
Sumatriptan-related

| | | | |
|---|---|---|---|
| Impurity A | 0.06 | 0.06 | 0.06 |
| Impurity B | 0.01 | 0.02 | 0.02 |
| Impurity C | BLD | BLD | BLD |
| Impurity D | 0.07 | 0.1 | 0.11 |

Naproxen-related

| | | | |
|---|---|---|---|
| Impurity E | 0.005 | 0.006 | 0.007 |
| Impurity F | BLD* | BLD | BLD |
| Impurity G | BLD | BLD | BLD |
| Impurity H | 0.001 | BLD | 0.001 |
| Total Impurities | 0.19 | 0.28 | 0.31 |

Dissolution (in 45 minutes)

| | | | |
|---|---|---|---|
| Sumatriptan | 100.6 | 99.5 | 99.6 |
| Naproxen | 101.6 | 100 | 97.6 |
| Water by KF | 4.9 | 5.1 | 4.5 |

*BLD: Below limit of detection.

A pharmacokinetic study is conducted. Pharmacokinetic parameters Cₘₐₓ (maximum concentration of drug in the plasma), AUC₀₋ₜ (area under the curve from the time of administration to the last time of a measurable plasma concentration), and AUC_{0-∝} (area under the curve from the time of administration to time infinity) are determined for tablets of Example 7 (test product, or “T”) and TREXIME® 85 mg/500 mg tablets (reference product, or “R”), in a crossover study involving 72 (fasting) and 67 (fed) subjects, with drug administered both in fasting and fed conditions. The average results for sumatriptan and naproxen are shown in Table 4.

TABLE 4

| | | Fasting | | | Fed | | |
|---|---|---|---|---|---|---|---|
| Sample and Drug | | Cₘₐₓ | AUC₀₋ₜ | AUC_{0-∝} | Cₘₐₓ | AUC₀₋ₜ | AUC_{0-∝} |
| Test | Sumatriptan | 75.5 | 306.5 | 314.9 | 49 | 257.6 | 267 |
| | Naproxen | 67.06 | 1302 | 1406.3 | 57.08 | 1134.3 | 1228.4 |
| Reference | Sumatriptan | 68.08 | 280.6 | 290.4 | 48.83 | 254.9 | 264.6 |
| | Naproxen | 65.49 | 1298.5 | 1397.3 | 56.43 | 1125.6 | 1222.7 |

### EXAMPLE 8

### Rapid-Disintegrating Monolithic Tablet comprising Sumatriptan and Naproxen

| | |
|---|---|
| Ingredient | Percent |

Granules 1

| | |
|---|---|
| Sumatriptan succinate | 11.84 |
| Sodium starch glycolate | 1.19 |
| Anhydrous lactose | 17.4 |
| Water* | q.s. |

Granules 2

| | |
|---|---|
| Naproxen sodium | 49.7 |
| Microcrystalline cellulose | 5.27 |
| Povidone K-30 | 2.4 |
| Water* | q.s. |
| Sodium starch glycolate | 1.2 |

Extragranular

| | |
|---|---|
| Microcrystalline cellulose | 5.3 |
| Sodium starch glycolate | 1.2 |
| Croscarmellose sodium | 1.3 |
| Talc | 2.7 |
| Magnesium stearate | 0.5 |

*Evaporates during processing.

Manufacturing Process:

Granules 1.

1) Sift sumatriptan succinate sodium starch glycolate, and anhydrous lactose through an ASTM #40 mesh sieve and mix.

2) Granulate step 1) materials using water.

3) Dry the granules in a dryer until loss on drying is not more than 2% at 60° C.

4) Sift the granules of step 4) through an ASTM #20 mesh sieve.

Granules 2.

1) Sift naproxen sodium, microcrystalline cellulose and sodium starch glycolate through an ASTM #40 mesh sieve.

2) Place step 1) materials into a granulator and dry mix.

3) Dissolve povidone in water to form a binder solution.

4) Granulate step 2) materials using step 3) binder solution.

5) Dry the granules in a dryer until the loss on drying is not more than 2% w/w at 60° C.

6) Sift the granules through an ASTM #20 mesh sieve.

Blending.

1) Mix granules 1 and granules 2.

2) Sift extragranular microcrystalline cellulose, sodium starch glycolate, and croscarmellose sodium through an ASTM #40 mesh sieve.

3) Blend step 1) materials with step 2) materials for about 10 minutes.

4) Sift talc and magnesium stearate through an ASTM #80 mesh sieve.

5) Blend step 3) with step 4) materials for about 5 minutes.

Compression.

1) Compress the final lubricated blend into a single layered tablet.

### EXAMPLE 9

### Naproxen Tablet Coated with Sumatriptan Layer

| | |
|---|---|
| Ingredient | Percent |

Naproxen Blend

| | |
|---|---|
| Naproxen sodium | 60.64 |
| Microcrystalline cellulose | 6.42 |
| Povidone K-30 | 2.86 |
| Water* | q.s. |
| Microcrystalline cellulose | 6.43 |
| Croscarmellose sodium | 1.58 |
| Talc | 3.28 |
| Magnesium stearate | 0.6 |

Seal Coating

| | |
|---|---|
| Hydroxypropyl methylcellulose (HPMC 5 cps) | 2.2 |
| Water* | q.s. |

Outer Coating

| | |
|---|---|
| Sumatriptan succinate | 14.4 |
| Povidone K-30 | 0.73 |
| Talc | 0.43 |
| Magnesium stearate | 0.43 |
| Water* | q.s. |

*Evaporates during processing.

Manufacturing Process:

1) Prepare a naproxen blend according to Example 2.

2) Compress the naproxen blend into a tablet.

3) Seal-coat the naproxen tablet of step 2) using HPMC 5 cps solution in water.

4) Dissolve sumatriptan succinate and povidone K-30 in water.

5) Disperse talc and magnesium stearate in the step 4) solution.

6) Coat the naproxen tablet of step 3) using the step 5) dispersion.

### EXAMPLE 10

### Bilayer Tablet Comprising Sumatriptan 119 mg and Naproxen 500 mg

| | |
|---|---|
| Ingredient | Grams |

Sumatriptan Layer

| | |
|---|---|
| Sumatriptan succinate | 119 |
| Dibasic calcium phosphate (Anhydroux | 119 |
| Emcompress) | |
| Croscarmellose sodium | 20 |
| Microcrystalline cellulose (Avicel PH102) | 98 |
| Sodium bicarbonate | 40 |
| Magnesium stearate | 4 |

Naproxen Layer

| | |
|---|---|
| Naproxen sodium | 500 |
| Microcrystalline cellulose (Avicel PH102) | 54 |
| Povidone K 90 | 40.5 |
| Water* | 450 |
| Sumatriptan granules from Layer 1 | 38.7 |
| Sodium bicarbonate | 20 |
| Microcrystalline cellulose (Avicel PH102) | 20 |
| Crospovidone | 31 |
| Colloidal silicon dioxide | 7 |
| Sodium stearyl fumarate | 14 |
| Magnesium stearate | 7 |

*Evaporates during processing.

Manufacturing Process:

Layer 1.

1) Sumatriptan succinate, dibasic calcium phosphate, and croscarmellose sodium are sifted through an ASTM #40 mesh sieve and dry mixed in a granulator for about 5 minutes.

2) Step 1) materials are granulated with water.

3) The wet granules are dried for about 1 hour until loss on drying at 65±10C is less than 2% w/w.

4) Avicel PH102 and sodium bicarbonate are sifted through an ASTM #40 mesh sieve, added to the above granules and blended for about 10 minutes.

5) Magnesium stearate is sifted through an ASTM #60 mesh sieve, added to the step 4) blend, and blended for about 5 minutes.

Layer 2:

1) Naproxen and Avicel PH102 are sifted through an ASTM #30 mesh sieve and placed into a fluid bed processor.

2) Povidone is dissolved in water and sprayed over the blend in the fluid bed processor at an inlet air temperature of about 53-56° C., followed by drying.

3) Sodium bicarbonate, Avicel PH102, crospovidone, and colloidal silicon dioxide are sifted through an ASTM #40 mesh sieve. The sifted materials and a portion of sumatriptan blend from Layer I are added to step 2) materials and blended for about 5 minutes.

4) Magnesium stearate is sifted through an ASTM #60 mesh sieve, added to step 3) blend and blended for about 5 minutes.

Compression:

732.2 mg of naproxen layer and 361.3 mg of sumatriptan layer are compressed together into a bilayer tablet.

The above compressed tablets are tested for dissolution characteristics in 900 mL of pH 6.8 phosphate buffer at 50 rpm stirring, in USP type I apparatus. The data are tabulated below.

Cumulative % of
Drug Dissolved

| Minutes | Sumatriptan | Naproxen |
|---|---|---|
| 5 | 90 | 17 |
| 15 | 94 | 45 |
| 30 | 96 | 78 |
| 45 | 95 | 97 |
| 60 | 96 | 100 |

### EXAMPLES 11-12

### Bilayer Tablets Comprising Sumatriptan 119 mg and Naproxen 500 mg

Grams

| | | |
|---|---|---|
| Ingredient | 11 | 12 |

Sumatriptan Granules

| | | |
|---|---|---|
| Sumatriptan succinate | 99.3 | 117 |
| Dibasic calcium phosphate | 99.3 | 117 |
| Microcrystalline cellulose (Avicel PH102) | 98.51 | 98.32 |
| Croscarmellose sodium | 19.7 | 19.66 |
| Sodium bicarbonate | 39.41 | 39.33 |
| Magnesium stearate | 1.97 | 1.97 |

Naproxen Granules

| | | |
|---|---|---|
| Naproxen sodium | 187.5 | 187.5 |
| Sodium bicarbonate | 12.5 | 12.5 |
| Sodium starch glycolate | 6.25 | 6.25 |
| Crosscarmellose sodium | 15.6 | 15.6 |
| Microcrystalline cellulose (Avicel PH102) | 33.8 | 33.8 |
| Povidone K90 | 26.25 | 26.25 |
| Naproxen sodium | 125 | 125 |
| Methanol* | 750 | 750 |
| Microcrystalline cellulose (Avicel PH 102) | 5 | 5 |
| Croscarmellose sodium | 10.5 | 10.5 |
| Colloidal silicon dioxide | 2 | 2 |
| Sodium stearyl fumarate | 13.5 | 13.5 |
| Magnesium stearate part 2 | 3.5 | 3.5 |

*Evaporates during processing.

Manufacturing Process:

A. Sumatriptan Succinate Granules:

1. Sumatriptan succinate and dicalcium phosphate are blended in a rapid mixer granulator for 5 minutes.

2. The blend of step 1 is granulated using 285 g of water in 5 minutes with impeller at fast speed and chopper at slow speed.

3. Granules are dried at 60° C. until loss on drying is less than 2% w/w.

4. Step 3 granules are sifted through an ASTM #30 mesh sieve and mixed with remaining ingredients, except magnesium stearate, for 5 minutes.

5. The step 4 mixture is blended with magnesium stearate for 5 minutes.

B. Naproxen Sodium Granules:

1. Sodium bicarbonate, sodium starch glycolate, croscarmellose sodium, Avicel PH1 02, and naproxen sodium (first quantity) are sifted together through an ASTM #30 mesh sieve and are blended in a polyethylene bag.

2. Naproxen sodium (second quantity) and PVP K90 are dissolved in methanol.

3. Step 1 blend is granulated with step 2 solution using a fluid bed processor (FBP) with top spray.

4. After granulation, drying is carried out in the FBP to obtain a loss on drying not more than 2.5% w/w.

5. Step 4 granules are sifted through an ASTM #22 mesh sieve and mixed with microcrystalline cellulose and croscarmellose sodium (second quantity) for 5 minutes.

6. Step 5 mixture is blended with colloidal silicon dioxide, sodium stearyl fumarate, and magnesium stearate for 5 minutes.

C. Compression:

705 mg of naproxen granules and 422 mg of sumatriptan granules are compressed together into a bilayer tablet.

The tablets of Examples 11 and 12 are subjected to dissolution testing in 900 ml of pH 6.8 phosphate buffer, at 50 rpm stirring in USP type I apparatus. The data are tabulated below.

| | Cumulative % of Drug Dissolved | | | |
|---|---|---|---|---|
| | 11 | | 12 | |
| Minutes | Sumatriptan | Naproxen | Sumatriptan | Naproxen |
| 10 | 90 | 34 | 92 | 31 |
| 15 | 90 | 49 | 96 | 46 |
| 30 | 91 | 86 | 99 | 82 |
| 45 | 91 | 100 | 100 | 100 |
| 60 | 92 | 101 | 100 | 100 |

### EXAMPLE 13

### Monolithic Tablets Comprising 119 mg of Sumatriptan Succinate and 500 mg of Naproxen Sodium

| | |
|---|---|
| Ingredient | Grams |

Sumatriptan Granules

| | |
|---|---|
| Sumatriptan succinate | 119 |
| Anhydrous Emcompress | 119 |
| Water* | 285 |

Naproxen Granules

| | |
|---|---|
| Naproxen sodium | 500 |
| Avicel PH102 (MCC) | 54 |
| Povidone | 27 |
| Water* | 550 |

Extragranular

| | |
|---|---|
| Avicel PH102 | 98.32 |
| Croscarmellose sodium (CCS) | 19.66 |
| Sodium bicarbonate | 39.33 |
| Magnesium stearate | 1.97 |

*Evaporates during processing.

Manufacturing Process:

Sumatriptan Granules

1) Sumatriptan succinate and Anhydrous Emcompress are blended in a granulator for about 5 minutes.

2) The mixture of step 1 is granulated using water.

3) Granules are dried at 65° C.±10° C. until loss on drying at 65° C.±5° C. is less than 2% w/w.

Naproxen Granules:

1) Naproxen and MCC are dry mixed for about 5 minutes in a granulator.

2) Povidone is dissolved in water.

3) Step 1) materials are granulated using povidone solution from step 2).

4) The wet granules are dried at 60° C. for about 15 minutes and milled through a 4 mm screen, followed by milling through a 2 mm screen.

5) The granules are further dried for another hour at 60° C. until loss on drying at 65° C.±10° C. is less than 2% w/w.

Blending:

1) Avicel PH102, CCS, and sodium bicarbonate are sifted through an ASTM #40 mesh sieve.

2) Sumatriptan granules, naproxen granules and step 1) materials are blended for about 15 minutes.

3) Magnesium stearate is sifted through an ASTM #60 mesh sieve, added to step 2) materials and blended for another 5 minutes.

4) The lubricated blend of step 3) is compressed into monolithic tablets having an average weight of 1011 mg.

We claim:

1. An inlay tablet, comprising an inner tablet and an outer tablet encompassing all but at least a portion of one surface of the inner tablet, the inner tablet comprising a first active pharmaceutical ingredient which is a triptan or a nonsteroidal anti-inflammatory drug, and the outer tablet comprising a second active pharmaceutical ingredient which is a triptan or a nonsteroidal anti-inflammatory drug, and wherein the first and the second active pharmaceutical ingredients are not both triptans or both nonsteroidal anti-inflammatory drugs.

2. The inlay tablet of claim 1, wherein the first active pharmaceutical ingredient is a triptan and the second active pharmaceutical ingredient is a nonsteroidal anti-inflammatory drug.

3. The inlay tablet of claim 1, wherein the first active pharmaceutical ingredient is sumatriptan or a salt thereof and the second active pharmaceutical ingredient is naproxen or a salt thereof.

4. The inlay tablet of claim 1, wherein the first active pharmaceutical ingredient is sumatriptan succinate and the second active pharmaceutical ingredient is naproxen sodium.

5. The inlay tablet of claim 1, wherein the first active pharmaceutical ingredient is a nonsteroidal anti-inflammatory drug and the second active pharmaceutical ingredient is a triptan.

6. The inlay tablet of claim 1, wherein the second active pharmaceutical ingredient is sumatriptan or a salt thereof and the first active pharmaceutical ingredient is naproxen or a salt thereof.

7. The inlay tablet of claim 1, wherein the second active pharmaceutical ingredient is sumatriptan succinate and the first active pharmaceutical ingredient is naproxen sodium.

8. The inlay tablet of claim 7, wherein 10 percent or more of the total weight of sumatriptan succinate present in the tablet is disposed in an inner tablet together with substantially all of the naproxen sodium.

9. A compressed layered tablet, comprising a first layer comprising substantially all of a first active pharmaceutical ingredient present in the tablet and at least 10%, but less than 90%, of the total amount of a second active pharmaceutical ingredient present in the tablet, with the balance of the second active pharmaceutical ingredient disposed in a second layer, the first and the second layers being arranged side-by-side, and wherein the first and second active pharmaceutical ingredients are triptans and nonsteroidal anti-inflammatory drugs, and when the first active pharmaceutical ingredient is a triptan, the second active pharmaceutical ingredient is a nonsteroidal anti-inflammatory drug, and when the first active pharmaceutical ingredient is a nonsteroidal anti-inflammatory drug, the second active pharmaceutical ingredient is a triptan.

10. The compressed layered tablet of claim 9, wherein the tablet is a mini-tablet.

11. The compressed layered tablet of claim 9, wherein a triptan is sumatriptan or a salt thereof, and a nonsteroidal anti-inflammatory drug is naproxen or a salt thereof.

12. The compressed layered tablet of claim 9, wherein the layers are arranged such that one layer completely surrounds the other.

13. The compressed layered tablet of claim 12, wherein a triptan is sumatriptan or a salt thereof and a nonsteroidal anti-inflammatory drug is naproxen or a salt thereof.

14. The compressed layered tablet of claim 9, wherein the layers are arranged to form an inlay tablet.

15. The compressed layered tablet of claim 14, wherein a triptan is sumatriptan or a salt thereof, and a nonsteroidal anti-inflammatory drug is naproxen or a salt thereof.

16. The compressed layered tablet of claim 9, wherein the triptan or nonsteroidal anti-inflammatory drug, which is present in both the first and second layers, is present in the first layer in an amount of from about 15 percent to about 80 percent of the total amount of that drug present in the tablet.

17. The compressed layered tablet of claim 16, wherein the triptan is sumatriptan or a salt thereof, present in an amount between about 1 and about 300 mg, calculated as the sumatriptan free base equivalent, and wherein the nonsteroidal anti-inflammatory drug is naproxen or a salt thereof, present in an amount between about 250 and about 550 mg, calculated as the naproxen free base equivalent.

18. The compressed layered tablet of claim 16, wherein the triptan is disposed in both the first and second layers.

19. The compressed layered tablet of claim 16, wherein the nonsteroidal anti-inflammatory drug is disposed in both the first and second layers.

20. The compressed layered tablet of claim 19, wherein the triptan is sumatriptan or as salt thereof and the nonsteroidal anti-inflammatory drug is naproxen or a salt thereof.

21. The compressed layered tablet of claim 19, wherein the triptan is sumatriptan succinate and the nonsteroidal anti-inflammatory drug is naproxen sodium.

22. The compressed layered tablet of claim 17, wherein more than about 70 percent of contained sumatriptan is released within about 45 minutes after immersion in 900 mL of pH 6.8 phosphate buffer in USP type I apparatus, with 50 rpm stirring.

23. The compressed layered tablet of claim 17, wherein more than about 70 percent of contained naproxen is released within about 45 minutes after immersion in 900 ml of pH 6.8 phosphate buffer in USP type I apparatus, with 50 rpm stirring.

24. The compressed layered tablet of claim 17, wherein total impurities are less than about 2 percent by weight of the total sumatriptan and naproxen content.

25. A compressed layered tablet or mini-tablet, comprising naproxen sodium and sumatriptan succinate, wherein a first layer comprises substantially all of the naproxen sodium present in the tablet or mini-tablet and about 13 percent by weight of the sumatriptan succinate present therein, and a second layer comprises the balance of the sumatriptan succinate, the layers being arranged side-by-side.
